(19) United States
(12) Patent Application Publication
(10) Pub. No.: US 2022/0409264 A1
(43) Pub. Date: Dec. 29, 2022

(54) ANTI-BACKDRIVE MECHANISM FOR VESSEL SEALING INSTRUMENT

(71) Applicant: Covidien LP, Mansfield, MA (US)

(72) Inventors: Grant T. Sims, Boulder, CO (US); William R. Whitney, Boulder, CO (US); Jason G. Weihe, Longmont, CO (US); James D. Allen, IV, Broomfield, CO (US); Jason T. Sanders, Longmont, CO (US); Alyssa M. Sawyer, Broomfield, CO (US); Curtis M. Siebenaller, Frederick, CO (US)

(21) Appl. No.: 17/849,014

(22) Filed: Jun. 24, 2022

Related U.S. Application Data
(60) Provisional application No. 63/214,928, filed on Jun. 25, 2021.

(51) Int. Cl. A61B 18/14; A61B 18/12

(52) U.S. Cl. CPC A61B 18/1447; A61B 18/1206; A61B 2018/0063

(57) ABSTRACT

A vessel sealing instrument includes a housing having a shaft extending from a distal end thereof including an end effector assembly having opposing first and second jaw members operably coupled thereto. One of the jaw members moveable between open and closed positions for clamping tissue with a closure pressure within the range of about 3 kg/cm² to about 16 kg/cm². The jaw members are adapted to connect to a generator for providing energy thereto in accordance with a sealing algorithm. An anti-backdrive mechanism is associated with the end effector assembly and includes: a drive shaft coupled to a controller and a screw on opposite ends, the screw configured to engage one of the jaw members upon extension thereof to provide additional closure pressure therebetween. The drive shaft is rotatable by the controller to extend the screw in response to tissue expansion during sealing based on the sealing algorithm.

### CROSS REFERENCE TO RELATED APPLICATION

This application claims the benefit of U.S. Provisional Application Ser. No. 63/214,928 filed Jun. 25, 2021, the entire contents of which being incorporated by reference herein.

### FIELD

The present disclosure relates to surgical instruments and, more particularly, to anti-backdrive mechanisms for vessel sealing instruments configured to maintain closure pressure during sealing.

### BACKGROUND

A surgical forceps is a pliers-like surgical instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is grasped under a closure pressure suitable to seal vessels or tissue, the actuation mechanism (e.g., handle) is locked during the delivery of electrosurgical energy to produce a seal. In some instance the surgeon holds the actuation mechanism during electrosurgical activation. During sealing, the tissue naturally expands against the closure pressure which, in some instances, can affect the resulting tissue seal as the closure pressure no longer falls within a particular closure pressure range.

Accordingly, there exists a need to maintain the closure pressure within the desired closure pressure range during the sealing process.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm². One or both of the first or second jaw members is adapted to connect to a generator configured to provide electrosurgical energy thereto in accordance with a sealing algorithm upon activation thereof.

An anti-backdrive mechanism is operably associated with the end effector assembly and includes a drive shaft operably coupled at a proximal end to a controller and at a distal end to a screw. The screw is configured to operably engage one (or both) of the first and second jaw members upon extension thereof to provide additional closure pressure between the jaw members. The drive shaft is selectively rotatable by the controller to extend the screw in response to tissue expansion during sealing based on the sealing algorithm.

In aspects according to the present disclosure, upon rotation of the drive shaft in a first direction, the screw engages an abutting surface disposed on a proximal end of the first jaw member. In other aspects according to the present disclosure, the first jaw member is rotatable relative to the second jaw member about a pivot and wherein the abutting surface is offset relative to the pivot.

In aspects according to the present disclosure, upon rotation of the drive shaft in a second direction, the screw retracts relative to the first jaw member allowing the first jaw member to open relative to the second jaw member. In other aspects according to the present disclosure, the controller rotates the drive shaft to extend the screw in response to tissue expansion during sealing based on the sealing algorithm, the screw configured to maintain the closure pressure between jaw members within the range of about 3 kg/cm² to about 16 kg/cm².

In accordance with other aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm².

An anti-backdrive mechanism is operably associated with the end effector assembly and includes a drive shaft operably coupled at a proximal end to a controller and at a distal end to a screw. The screw is configured to operably engage one (or both) of the first and second jaw members upon extension thereof to provide additional closure pressure between the jaw members. The controller is configured to continually monitor the closure pressure between the jaw members. The drive shaft is selectively rotatable by the controller to extend the screw in response to the closure pressure falling outside the range of about 3 kg/cm² to about 16 kg/cm².

In aspects according to the present disclosure, upon rotation of the drive shaft in a first direction, the screw engages an abutting surface disposed on a proximal end of the first jaw member. In other aspects according to the present disclosure, the first jaw member is rotatable relative to the second jaw member about a pivot and wherein the abutting surface is offset relative to the pivot.

In aspects according to the present disclosure, upon rotation of the drive shaft in a second direction, the screw retracts relative to the first jaw member allowing the first jaw member to open relative to the second jaw member.

In aspects according to the present disclosure, the controller is configured to continually monitor the closure pressure between the jaw members when the surgical instrument is activated to seal tissue and wherein the controller rotates the drive shaft to extend the screw in response to the closure pressure falling outside the range of about 3 kg/cm² to about 16 kg/cm².

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm². One or both of the first or second jaw members is adapted to connect to a generator configured to provide electrosurgical energy thereto in accordance with a sealing algorithm upon activation thereof.

An anti-backdrive mechanism is operably associated with the end effector assembly and includes a drive shaft operably coupled at a proximal end to a controller and at a distal end to a camming wedge. The camming wedge is configured to operably engage one (or both) of the first and second jaw members upon extension thereof to provide additional closure pressure between the jaw members. The drive shaft is selectively translatable by the controller to move the camming wedge in response to tissue expansion during sealing based on the sealing algorithm to increase the closure pressure.

In aspects according to the present disclosure, upon translation of the drive shaft, the camming wedge engages an abutting surface disposed on a proximal end of the first jaw member. In other aspects according to the present disclosure, the first jaw member is rotatable relative to the second jaw member about a pivot and wherein the abutting surface is offset relative to the pivot.

In aspects according to the present disclosure, the controller is configured to continually monitor the closure pressure between the jaw members when the surgical instrument is activated to seal tissue and wherein the controller translates the drive shaft to move the camming wedge against the abutting surface in response to the closure pressure falling outside the range of about 3 kg/cm² to about 16 kg/cm². In other aspects according to the present disclosure, the abutting surface is angled to complement the angle of the camming wedge.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm². One or both of the first or second jaw members is adapted to connect to a generator configured to provide electrosurgical energy thereto in accordance with a sealing algorithm upon activation thereof. One or both of the first or second jaw members includes integrated carbon disposed therein configured to increase the rigidity thereof to resist expansion forces during sealing.

Provided in accordance with aspects of the present disclosure is a vessel sealing instrument including a housing having a shaft extending from a distal end thereof. A distal end of the shaft includes an end effector assembly having a pair of opposing first and second jaw members operably coupled thereto. One or both of the first or second jaw members is moveable between an open position and a closed position for clamping tissue with a closure pressure within the range of about 3 kg/cm² to about 16 kg/cm². One or both of the first or second jaw members is encapsulated with a liquid metal, alloy of carbide structure to increase the rigidity thereof to resist expansion forces during sealing.

In aspects according to the present disclosure, the one or both jaw members is encapsulated by spraying, dipping or casting.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of an electrosurgical forceps provided in accordance with the present disclosure having in-line electrosurgical activation;

FIG. 1B is a perspective view of an electrosurgical forceps provided in accordance with another embodiment of the present disclosure having a ratchet-like handle assembly;

FIG. 2A is an enlarged, perspective view of an end effector assembly of the electrosurgical forceps of FIG. 1 wherein first and second jaw members of the end effector assembly are disposed in a spaced-apart position;

FIG. 2B is an enlarged, perspective view of the end effector assembly of FIG. 2A wherein the first and second jaw members are disposed in an approximated position;

FIG. 3A is a side view of a proximal portion of the electrosurgical forceps of FIG. 1 with a movable handle and trigger thereof disposed in respective un-actuated positions;

FIG. 3B is a side view of the proximal portion of the electrosurgical forceps shown in FIG. 3A with the movable handle disposed in an actuated position and the trigger disposed in the un-actuated position;

FIG. 3C is a side view of the proximal portion of the electrosurgical forceps shown in FIG. 3A with the movable handle and trigger disposed in respective actuated positions;

FIG. 4A is a side view of another proximal portion of the electrosurgical forceps of FIG. 1 with portions removed to illustrate a trigger assembly thereof with the trigger disposed in the un-actuated position;

FIG. 4B is a side view of the proximal portion of the electrosurgical forceps shown in FIG. 4A with portions removed to illustrate the trigger assembly with the trigger disposed in the actuated position;

FIG. 5A is an enlarged, side view of an end effector assembly having a screw-like anti-backdrive mechanism provided in accordance with one embodiment of the present disclosure;

FIG. 5B is an enlarged, side view of an end effector assembly having a wedge-like anti-backdrive mechanism provided in accordance with another embodiment of the present disclosure; and

FIG. 5C is an enlarged, side view of an end effector assembly having a passive anti-backdrive mechanism provided in accordance with another embodiment of the present disclosure.

### DETAILED DESCRIPTION

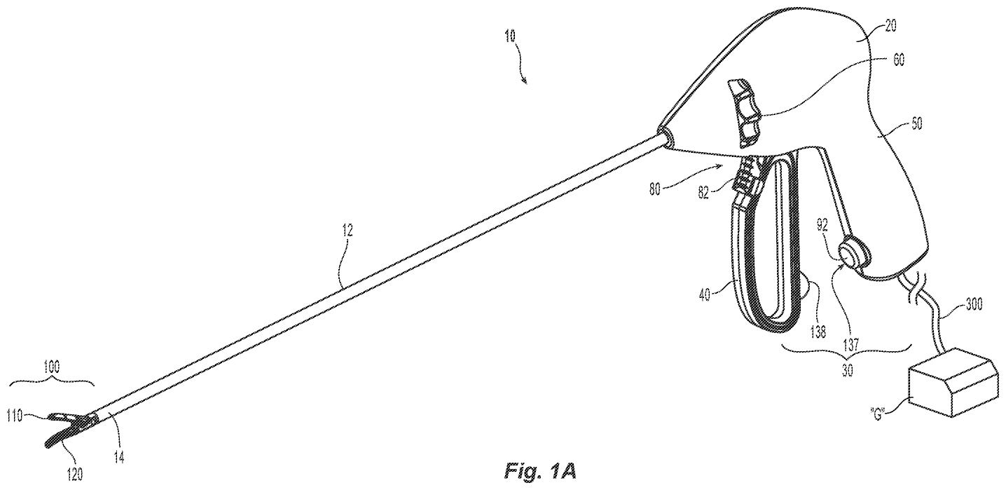

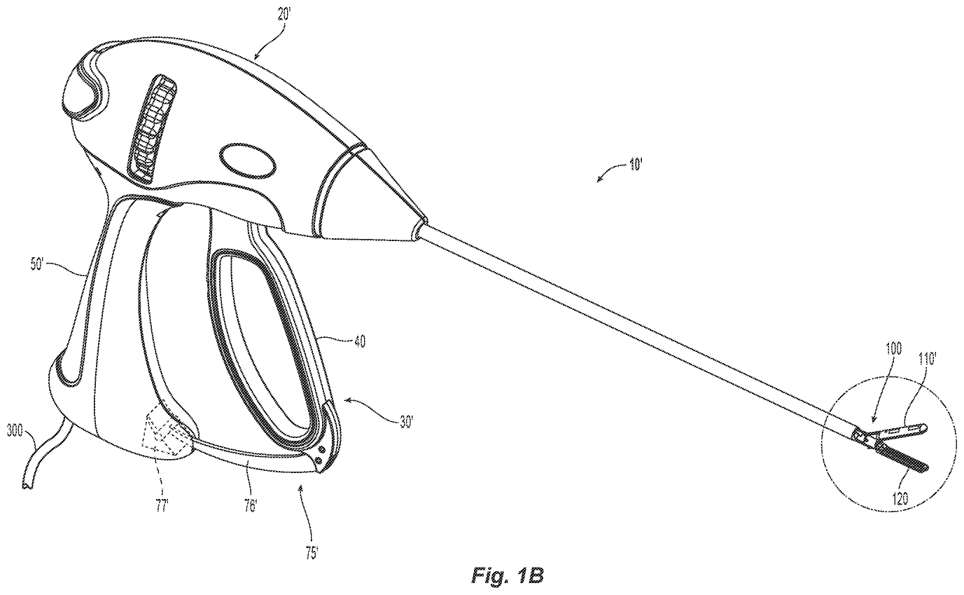

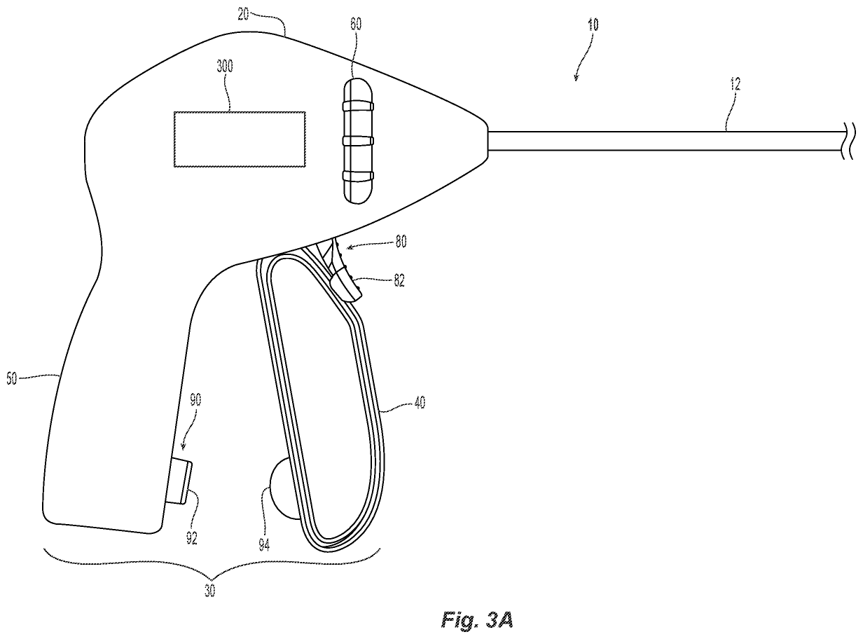

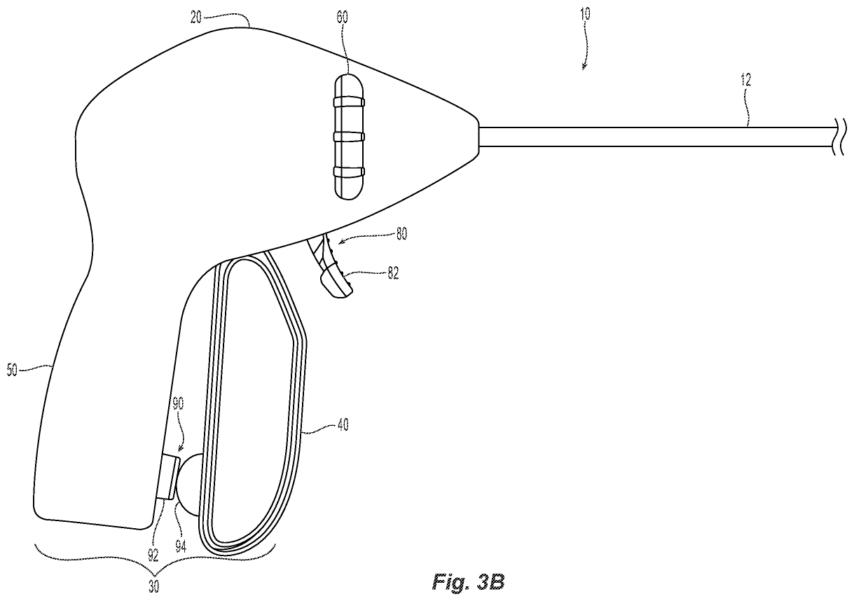

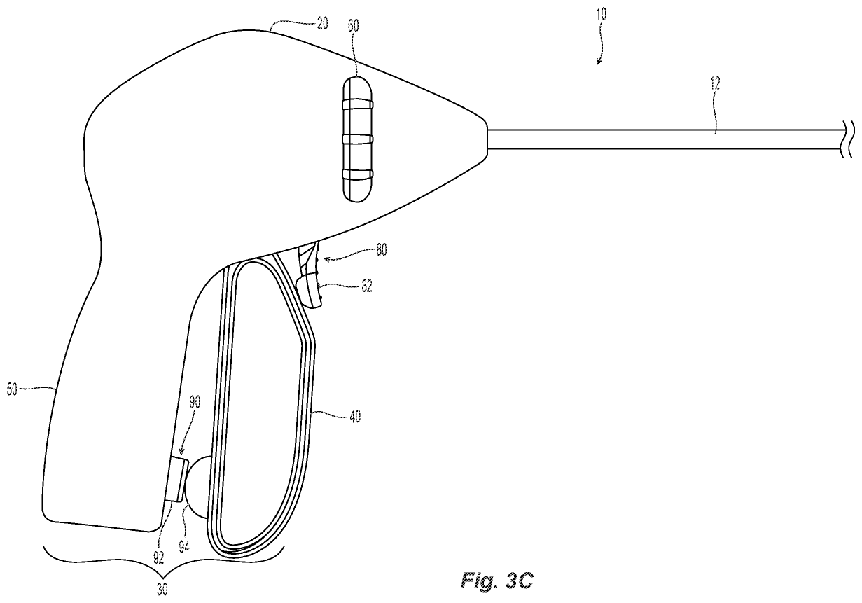

Referring to FIG. 1A, a surgical instrument provided in accordance with the present disclosure is shown configured as a bipolar electrosurgical forceps 10 for use in connection with endoscopic surgical procedures, although the present disclosure may be equally applicable for use with other surgical instruments such as those for use in endoscopic and/or traditional open surgical procedures. Forceps 10 generally includes a housing 20, a handle assembly 30, a rotating assembly 60, a trigger assembly 80, an activation assembly 90 (See FIGS. 3A-3C), and an end effector assembly 100 including first and second jaw members 110, 120.

Forceps 10 further includes a shaft 12 having a distal end portion 14 configured to engage (directly or indirectly) end effector assembly 100 and a proximal end portion 16 that engages (directly or indirectly) housing 20. Rotating assembly 60 is rotatable in either direction to rotate shaft 12 and end effector assembly 100 relative to housing 20 in either direction. Housing 20 houses the internal working components of forceps 10.

An electrosurgical cable 300 connects forceps 10 to an electrosurgical generator “G” or other suitable energy source, although forceps 10 may alternatively be configured as a handheld instrument incorporating energy-generating and/or power components thereon or therein. Cable 300 includes wires (not shown) extending therethrough, into housing 20, and through shaft 12, to ultimately connect electrosurgical generator “G” to jaw member 110 and/or jaw member 120 of end effector assembly 100. Activation button 92 of activation assembly 90 is disposed on housing 20 are electrically coupled between end effector assembly 100 and cable 300 to enable the selective supply of energy to jaw member 110 and/or jaw member 120, e.g., upon activation of activation button 92. However, other suitable electrical connections and/or configurations for supplying electrosurgical energy to jaw member 110 and/or jaw member 120 may alternatively be provided, as may other suitable forms of energy, e.g., ultrasonic energy, microwave energy, light energy, thermal energy, etc.

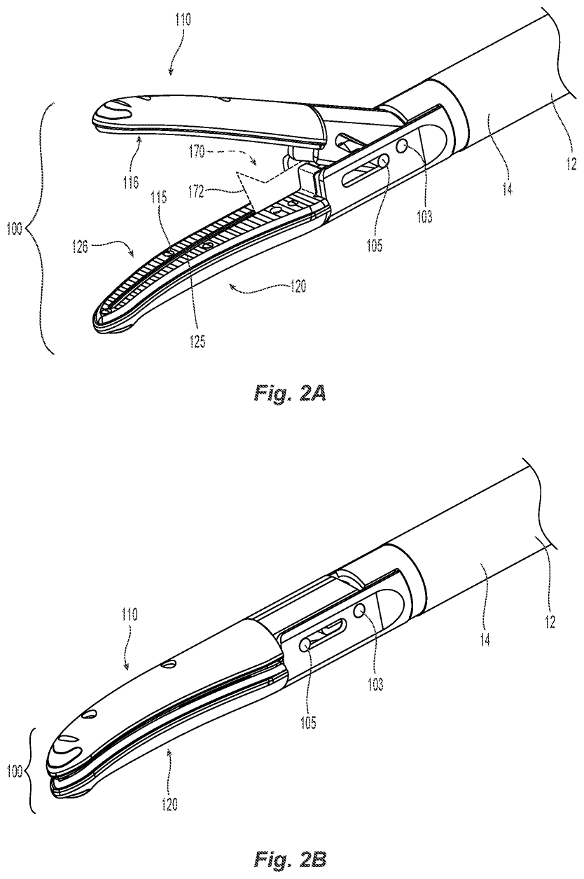

Forceps 10 additionally includes a knife assembly 170 (FIG. 2A) operably coupled to trigger assembly 80 and extending through housing 20 and shaft 12. One or both of jaw members 110, 120 defines a knife channel 125 (FIG. 2A) configured to permit reciprocation of a knife blade 172 (FIG. 2A) of knife assembly 170 (FIG. 2A) therethrough, e.g., in response to actuation of trigger 82 of trigger assembly 80. Trigger assembly 80 is described in greater detail below as are other embodiments of trigger assemblies configured for use with forceps 10.

With additional reference to FIGS. 2A and 2B, end effector assembly 100, as noted above, is disposed at distal end portion 14 of shaft 12 and includes a pair of jaw members 110 and 120 pivotable between a spaced-apart position and an approximated position for grasping tissue therebetween. End effector assembly 100 is designed as a unilateral assembly, e.g., wherein one of the jaw members 120 is fixed relative to shaft 12 and the other jaw member 110 is movable relative to both shaft 12 and the fixed jaw member 120. However, end effector assembly 100 may alternatively be configured as a bilateral assembly, e.g., wherein both jaw member 110 and jaw member 120 are movable relative to one another and with respect to shaft 12.

Each jaw member 110, 120 of end effector assembly 100 includes an electrically-conductive tissue-contacting surface 116, 126. Tissue-contacting surfaces 116 are positioned to oppose one another for grasping and treating tissue. More specifically, tissue-contacting surfaces 116, 126 are electrically coupled to the generator “G,” e.g., via cable 300, and activation button 92 to enable the selective supply of energy thereto for conduction through tissue grasped therebetween, e.g., upon activation of activation button 92. One or both of tissue-contacting surfaces 116, 126 may include one or more stop members 115 extending therefrom to define a minimum gap distance between electrically-conductive tissue-contacting surfaces 116, 126 in the approximated position of jaw members 110, 120, facilitate grasping of tissue, and/or inhibit shorting between electrically-conductive tissue-contacting surfaces 116, 126.

The stop member(s) 115 may be formed at least partially from an electrically-insulative material or may be effectively insulative by electrically isolating the stop member(s) from one or both of the electrically-conductive tissue-contacting surfaces 116, 126. The one or more stop members 115 may be disposed on one or both jaw members 110, 120 or on the tissue-contacting surfaces 116, 126 and are configured to regulate the distance therebetween. Details relating to various stop member designs are disclosed in U.S. Patent No. 7,857,812, 10,687,887 the entire contents of each of which being incorporated by reference here.

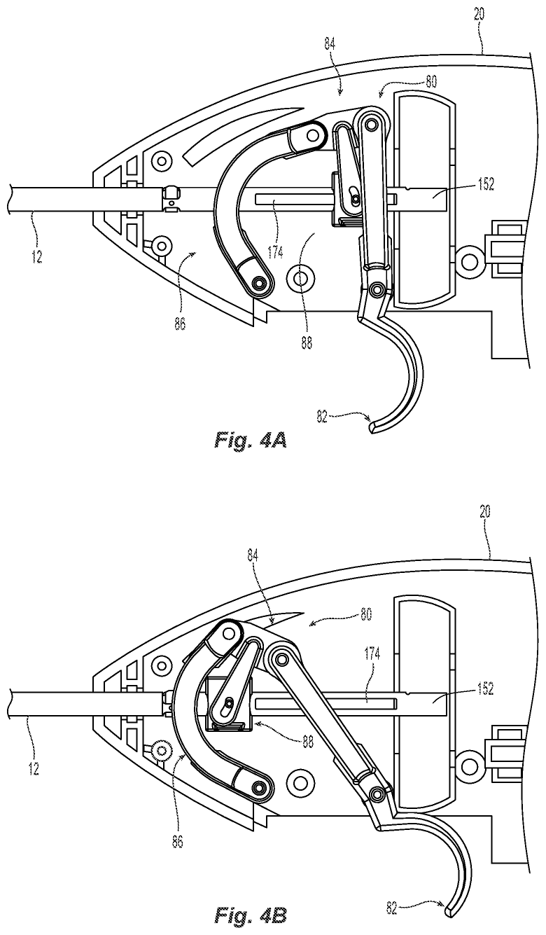

A pivot pin 103 of end effector assembly 100 extends transversely through aligned apertures defined within jaw members 110, 120 and shaft 12 to pivotably couple jaw member 110 to jaw member 120 and shaft 12. A cam pin 105 of end effector assembly 100 extends transversely through cam slots defined within jaw members 110, 120 and is operably engaged with a distal end portion of a drive bar 152 (FIGS. 4A and 4B) of a drive assembly 300 (only drive bar 152 (FIGS. 4A and 4B) of the drive assembly 300 is shown and drive assembly is generically represent by component 300 in FIG. 3A) such that longitudinal translation of drive bar 152 (FIGS. 4A and 4B) through shaft 12 translates cam pin 105 relative to jaw members 110, 120. Various drive assemblies are shown and described with respect to commonly-owned U.S. Pat. Nos. 7,857,812, 8,540,711, 7,384,420, 7,090,673, 7,101,372, 7,255,697, 7,101,371, 7,131,971, 7,083,618, and 10,842,553, the entire contents of each of which being incorporated by reference herein.

More specifically, distal translation of cam pin 105 relative to jaw members 110, 120 urges cam pin 105 distally through the cam slots to thereby pivot jaw members 110, 120 from the spaced-apart position towards the approximated position, although cam slots may alternatively be configured such that proximal translation of cam pin 105 pivots jaw members 110, 120 from the spaced-apart position towards the approximated position. One suitable drive assembly is described in greater detail, for example, in U.S. Pat. No. 9,655,673, the entire contents of which are hereby incorporated herein by reference.

Referring to FIGS. 1A-3C, handle assembly 30 includes a fixed handle 50 and an actuator, e.g., movable handle 40. Fixed handle 50 is integrally associated with housing 20 and movable handle 40 is movable relative to fixed handle 50. Movable handle 40 is ultimately connected to the drive assembly (not shown) that, together, mechanically cooperate to impart movement of jaw members 110 and 120 between the spaced-apart and approximated positions to grasp tissue between electrically-conductive surfaces 116, 126, respectively. More specifically, pivoting of movable handle 40 relative to fixed handle 50 from an un-actuated position towards an actuated position pivots jaw members 110, 120 from the spaced-apart position towards the approximated position. On the other hand, when movable handle 40 is released or returned towards the initial position relative to fixed handle 50, jaw members 110, 120 are returned towards the spaced-apart position.

A biasing spring (not shown) associated with movable handle 40 and/or the drive assembly may be provided to bias jaw members 110, 120 towards a desired position, e.g., the spaced-apart position or the approximated position. Various drive assemblies are shown and described in any one of the above-identified commonly-owned U.S. Patents referenced herein.

Fixed handle 50 operably supports activation button 92 of activation assembly 90 thereon in an in-line position, wherein activation button 92 is disposed in the actuation path of movable handle 40. In this manner, upon pivoting of movable handle 40 relative to fixed handle 50 from the actuated position to an activated position, protrusion 94 of movable handle 40 is urged into contact with activation button 92 to thereby activate activation button 92 and initiate the supply of energy to electrically-conductive surfaces 116, 126, e.g., to treat tissue grasped therebetween. Alternatively, actuation button 92 may be disposed in any other suitable position, on housing 20 or remote therefrom, to facilitate manual activation by a user to initiate the supply of energy to electrically-conductive surfaces 116, 126.

With reference to FIGS. 1A-2B and 4A-4B, as noted above, trigger assembly 80 is operably coupled to knife blade 172 of knife assembly 170. More specifically, trigger 82 of trigger assembly 80 is selectively actuatable, e.g., from an un-actuated position (FIGS. 3A and 4A) to an actuated position (FIGS. 3C and 4B), to deploy knife blade 172 distally through jaw members 110, 120 to cut tissue grasped between electrically-conductive surfaces 116, 126. Knife assembly 170 includes knife blade 172 and a knife bar 174 engaged with and extending proximally from knife blade 172 through shaft 12 and drive bar 152 into housing 20 where knife bar 174 is operably coupled with trigger assembly 80, as detailed below.

Referring to FIGS. 4A and 4B, trigger assembly 80 includes trigger 82, a link 84, e.g., a T-link 84, a link 86, e.g., an arcuate linkage 86 although other configurations, e.g., linear, angled, etc. are also contemplated, and a slider block 88. In this manner, trigger assembly 80 defines a four-bar mechanical linkage assembly for driving slider block 88 to actuate the knife blade 172. This and other types of trigger mechanism are also contemplated such as, for example, trigger mechanisms described in any one of the above-identified commonly-owned U.S. Patents referenced herein or U.S. patent application Ser. No. 16/558,477, the entire contents of each of which being incorporated by reference herein.

As mentioned above, pivoting of movable handle 40 relative to fixed handle 50 from an un-actuated position towards an actuated position pivots jaw members 110, 120 from the spaced-apart position towards the approximated position for grasping tissue therebetween. When fully grasped, the drive assembly 300 is configured to initially generate a closure pressure suitable for sealing vessels upon activation of electrosurgical energy from generator “G”. Maintaining closure pressures within the range of about 3 Kg/cm² to about 16 Kg/cm² are known to promote quality seals.

With in-line actuation instruments, the surgeon is typically required to maintain the handle 40 in position to continually maintain the closure pressure. For example and as shown in FIG. 1A, handle 40 is initially actuated under a light pressure to grasp and manipulate tissue prior to sealing as the jaw members 110, 120 may be closed without fully actuating handle 40 relative to handle 50. Once the tissue is properly positioned between jaw members 110, 120, the handle 40 may be fully actuated to close the jaw members 110, 120 about tissue within the above-noted pressure range and simultaneously activate the forceps 10 for sealing. With this type of forceps 10, the surgeon must maintain the handle 40 fully actuated to maintain the initial closure pressure. This is known as in-line activation.

Other forceps e.g., forceps 10′ of FIG. 1B include handle assemblies 30′ (including moveable handle 40′ and fixed handle 50′) that have a ratchet-like locking system 75′ affixed to a portion of the housing 20′ or handle assembly 30′ which is configured to lock handle 40′ relative to the fixed handle 50′ to initially generate and maintain the appropriate closure pressures between jaw members 110′, 120′ when locked. Ratchet-like locking system 75′ includes a flange 76′ extending from handle 40′ configured to mechanically engage and lock within a corresponding ramp 77′ (shown in phantom) disposed within handle 50′. Various such forceps and handle assemblies are shown in any one of the above-identified commonly-owned U.S. Patents referenced herein.

After the initial closure pressure within the above-identified range is generated and the jaw members 110, 120 (or 110′, 120′) are clamped on a vessel or on tissue, the forceps 10 (10′) is ready for activation. As mentioned above, during sealing the vessel or tissue expands against the jaw members, e.g., jaw members 110′, 120′, which may reduce the actual closure pressure during formation of the seal. If the closure pressure falls outside of the above-noted range, the seal may not be as effective.

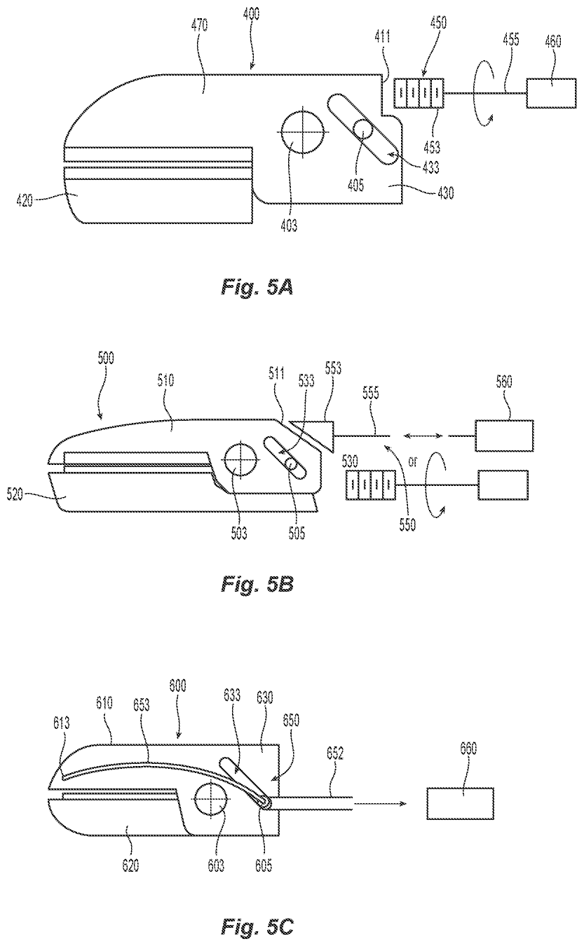

FIGS. 5A-5C show various end effector assemblies which include one or more so-called “anti-anti-backdrive assemblies” configured to maintain the required closure pressure during the sealing process. All of the below-described anti-anti-backdrive assemblies are configured to provide additional pressure to the jaw members 110, 120 to offset the forces attributed to tissue expansion. It is envisioned that any of the below-described anti-backdrive assemblies may be: passive, e.g., prevent the jaw members from 110, 120 from moving during tissue expansion; proactive, e.g., anticipate tissue expansion and counteract the same; or reactive, e.g., measure the expansion or rate of expansion (feedback) and counteract the same.

FIG. 5A shows one embodiment of an end effector assembly 400 having a jaw member 410 equipped with a anti-backdrive assembly 450. Anti-backdrive assembly 450 includes a drive shaft 455 operably coupled to a controller 460 at a proximal end thereof and operably coupled to an extendible screw 453 at a distal end thereof. Screw 453 is biased against the controller 460 (or some other surface) such that rotation thereof in a first direction extends the screw 453 relative to the surface, e.g., controller 460, and rotation in the opposite direction retracts the screw 453 relative to the surface, e.g., controller 460. The drive shaft 455 may be configured to rotate with the screw 453.

Jaw member 410 of end effector assembly 400 includes a rear flange 430 having an abutting surface 411 defined in a proximally-facing surface thereof configured to engage the screw 453 upon extension thereof. The abutting surface 411 is located above-center of the pivot 403 such that, after the jaw members 410, 420 are closed about tissue via actuation of pin 405 within cam slot 433 defined within the flange 430, the controller 460 extends the screw 453 into engagement with abutting surface 411 to further cam jaw member 410 about pivot 403 to increase closure pressure as needed. The controller 460 may extend the screw 453 upon: user request; based on a sealing algorithm; on reaction to feedback from the jaw members 410, 420 or tissue disposed therebetween; or in anticipation of tissue expansion during sealing.

The controller 460 may continually monitor the jaw members 410, 420 for feedback and adjust the screw 453 accordingly to maintain the appropriate closure pressure between the jaw members 410, 420 during the entire sealing process. Various types of sensors (not shown) or algorithms may be utilized for this purpose. Once sealed, the screw 453 is fully retracted to allow the jaw members 410, 420 to open via handle 40 (or 40′).

Turning now to FIG. 5B which shows another embodiment of an anti-backdrive mechanism 550 for use with forceps 10, 10′. Anti-backdrive mechanism 550 is similar to anti-backdrive mechanism 450 and, as such, only those differences will be discussed below. Anti-backdrive mechanism 550 includes a drive shaft 555 operably coupled to a controller 560 at a proximal end thereof and operably coupled to wedge 553 at a distal end thereof. Wedge 553 is extendible and retractable relative to controller 560 via actuation of the drive shaft 555.

Jaw member 510 of end effector assembly 500 includes a rear flange 530 having an angled surface 511 defined in a proximally-facing surface thereof configured to engage the wedge 553 upon extension thereof. The angled surface 511 is located above-center of the pivot 503 such that, after the jaw members 510, 520 are closed about tissue via actuation of pin 505 within cam slot 533 defined within the flange 530, the controller 560 extends the drive shaft 555 and wedge 553 into engagement with angled surface 511 to further cam jaw member 510 about pivot 503 to increase closure pressure. The controller 560 may extend the drive shaft 555 and wedge 553 upon: user request; based on a sealing algorithm; on reaction to feedback from the jaw members 510, 520 or tissue disposed therebetween; or in anticipation of tissue expansion during sealing.

Much like controller 460, controller 560 may continually monitor the jaw members 510, 520 for feedback and adjust the wedge 553 accordingly to maintain the appropriate closure pressure between the jaw members 510, 520 during the entire sealing process. Various types of sensors (not shown) or algorithms may be utilized for this purpose. Once sealed, the wedge 553 is fully retracted to allow the jaw members 510, 520 to open via handle 40 (or 40′).

FIG. 5C shows another embodiment of a anti-backdrive mechanism 650 for use with forceps 10, 10′. Anti-backdrive mechanism 650 is configured to cooperate with end effector assembly 600 having opposing jaw members 610, 620 configured to rotate about a pivot 603 upon actuation of drive rod 652. Drive rod 652 moves the jaw members 610, 620 between open and closed positions upon translation thereof by virtue of a cam pin 605 engaging a cam slot 633 defined in a proximal flange 630 of jaw member 610.

Anti-backdrive mechanism 650 includes a tension rod, tension cable or flat spring steel 653 (hereinafter “flat spring 653”) operably coupled at a proximal end to the cam pin 605 and fixed at a distal end thereof to a distal end of the jaw member 610. When the cam pin 605 is translated via drive rod 652 to close jaw members 610, 620 under an initial closure pressure within the above-identified closure pressure range, the flat spring 653 is moved into position to offset the forces associated with tissue expansion or resist the jaw members 610, 620 from opening or moving due to tissue expansion. As can be appreciated, this maintains the closure pressure within the appropriate ranges for consistent and effective tissue sealing. Once sealed, the drive rod 652 is fully retracted to allow the jaw members 610, 620 to open via handle 40 (or 40′) and the closure force associated with the flat spring 653 is released.

In embodiments, the drive rod 652 or cam pin (or the drive rods or cam pins with any aforementioned embodiment) may be actuated using the activation energy associated with the jaw members 610, 620 when sealing tissue. For example, energy may be diverted from the electrosurgical generator “G” to actuate the drive rod, e.g., drive rod 652, to resist the forces of expansion during sealing. Other devices may also be energized utilizing electrosurgical energy from the generator “G”, e.g., solenoids, sensors, or any of the above-identified anti-backdrive mechanisms or elements thereof.

Compared to the anti-backdrive mechanisms 450, 550 of FIGS. 5A and 5B, respectively, anti-backdrive mechanism 605 does not require feedback or continual monitoring during the sealing process. As such, anti-backdrive mechanism 650 acts more passively than the other aforementioned anti-backdrive mechanisms 450, 550 and only when the expansion forces associated with tissue sealing cause the closure pressure between jaw members 610, 620 to fall will anti-backdrive 650 work to counteract these forces to maintain the closure pressure within the appropriate closure pressure range.

In embodiments, the jaw members or jaw housings may be made from a carbide structure or integrated with a carbide material in fashion to resist the forces of tissue expansion during the sealing process. For example, jaw members 410, 420 (or any of the abovementioned jaw members) may be configured to include a carbide material integrated therein to significantly stiffen the jaw members 410, 420 to resist the forces associated with tissue expansion. Various configurations are envisioned such as matrix patterns, truss patterns or other known construction patterns.

In embodiments, the jaw members or jaw housings may be encapsulated with a liquid metal, alloy or carbide structure and allowed to cure to stiffen the jaw members. Various liquid metals, alloys and carbide structures are envisioned in varying thicknesses and may be configured to encapsulate the jaw members, e.g., jaw members 410, 420, by spraying, dipping, casting etc.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A vessel sealing instrument, comprising:
a housing having a shaft extending from a distal end thereof, a distal end of the shaft having an end effector assembly including a pair of opposing first and second jaw members operably coupled thereto, at least one of the first or second jaw members moveable between an open position and a closed position for clamping tissue with a closure pressure within the range of about 3 kg/cm² to about 16 kg/cm², at least one of the first or second jaw members adapted to connect to a generator configured to provide electrosurgical energy thereto in accordance with a sealing algorithm upon activation thereof; and
an anti-backdrive mechanism operably associated with the end effector assembly, the anti-backdrive mechanism including:
a drive shaft operably coupled at a proximal end to a controller and at a distal end to a screw, the screw configured to operably engage at least one of the first and second jaw members upon extension thereof to provide additional closure pressure between the jaw members, the drive shaft selectively rotatable by the controller to extend the screw in response to tissue expansion during sealing based on the sealing algorithm.

2. The vessel sealing instrument according to claim 1, wherein, upon rotation of the drive shaft in a first direction, the screw engages an abutting surface disposed on a proximal end of the first jaw member.

3. The vessel sealing instrument according to claim 2, wherein the first jaw member is rotatable relative to the second jaw member about a pivot and wherein the abutting surface is offset relative to the pivot.

4. The vessel sealing instrument according to claim 1, wherein, upon rotation of the drive shaft in a second direction, the screw retracts relative to the first jaw member allowing the first jaw member to open relative to the second jaw member.

5. The vessel sealing instrument according to claim 1, wherein the controller rotates the drive shaft to extend the screw in response to tissue expansion during sealing based on the sealing algorithm, the screw configured to maintain the closure pressure between jaw members within the range of about 3 kg/cm² to about 16 kg/cm².

6. A vessel sealing instrument, comprising:
a housing having a shaft extending from a distal end thereof, a distal end of the shaft having an end effector assembly including a pair of opposing first and second jaw members operably coupled thereto, at least one of the first or second jaw members moveable between an open position and a closed position for clamping tissue with a closure pressure within the range of about 3 kg/cm² to about 16 kg/cm²; and
an anti-backdrive mechanism operably associated with the end effector assembly, the anti-backdrive mechanism including:
a drive shaft operably coupled at a proximal end to a controller and at a distal end to a screw, the screw configured to operably engage at least one of the first and second jaw members upon extension thereof to provide additional closure pressure between the jaw members, the controller configured to continually monitor the closure pressure between the jaw members;
the drive shaft selectively rotatable by the controller to extend the screw in response to the closure pressure falling outside the range of about 3 kg/cm² to about 16 kg/cm².

7. The vessel sealing instrument according to claim 6, wherein, upon rotation of the drive shaft in a first direction, the screw engages an abutting surface disposed on a proximal end of the first jaw member.

8. The vessel sealing instrument according to claim 6, wherein the first jaw member is rotatable relative to the second jaw member about a pivot and wherein the abutting surface is offset relative to the pivot.

9. The vessel sealing instrument according to claim 6, wherein, upon rotation of the drive shaft in a second direction, the screw retracts relative to the first jaw member allowing the first jaw member to open relative to the second jaw member.

10. The vessel sealing instrument according to claim 6, wherein the controller is configured to continually monitor the closure pressure between the jaw members when the surgical instrument is activated to seal tissue and wherein the controller rotates the drive shaft to extend the screw in response to the closure pressure falling outside the range of about 3 kg/cm² to about 16 kg/cm².

11. A vessel sealing instrument, comprising:
a housing having a shaft extending from a distal end thereof, a distal end of the shaft having an end effector assembly including a pair of opposing first and second jaw members operably coupled thereto, at least one of the first or second jaw members moveable between an open position and a closed position for clamping tissue with a closure pressure within the range of about 3 kg/cm² to about 16 kg/cm², at least one of the first or second jaw members adapted to connect to a generator configured to provide electrosurgical energy thereto in accordance with a sealing algorithm upon activation thereof; and
an anti-backdrive mechanism operably associated with the end effector assembly, the anti-backdrive mechanism including:
a drive shaft operably coupled at a proximal end to a controller and at a distal end to a camming wedge, the camming wedge configured to operably engage at least one of the first and second jaw members upon extension thereof to provide additional closure pressure between the jaw members, the drive shaft selectively translatable by the controller to move the camming wedge in response to tissue expansion during sealing based on the sealing algorithm to increase the closure pressure.

12. The vessel sealing instrument according to claim 11, wherein, upon translation of the drive shaft, the camming wedge engages an abutting surface disposed on a proximal end of the first jaw member.

13. The vessel sealing instrument according to claim 12, wherein the first jaw member is rotatable relative to the second jaw member about a pivot and wherein the abutting surface is offset relative to the pivot.

14. The vessel sealing instrument according to claim 11, wherein the controller is configured to continually monitor the closure pressure between the jaw members when the surgical instrument is activated to seal tissue and wherein the controller translates the drive shaft to move the camming wedge against the abutting surface in response to the closure pressure falling outside the range of about 3 kg/cm² to about 16 kg/cm².

15. The vessel sealing instrument according to claim 11, wherein the abutting surface is angled to complement the angle of the camming wedge.
